(19) United States
(12) Patent Application Publication
(10) Pub. No.: US 2017/0367617 A1
(43) Pub. Date: Dec. 28, 2017

(54) PROBABILISTIC NON-INVASIVE ASSESSMENT OF RESPIRATORY MECHANICS FOR DIFFERENT PATIENT CLASSES

(71) Applicant: KONINKLIJKE PHILIPS N.V., EINDHOVEN (NL)

(72) Inventors: ANTONIO ALBANESE, NEW YORK, NY (US); NICOLAS WADIH CHBAT, WHITE PLAINS, NY (US); ADAM JACOB SEIVER, LOS ALTOS HILLS, CA (US)

(21) Appl. No.: 15/535,444

(22) PCT Filed: Dec. 16, 2015
(86) PCT No.: PCT/IB2015/059683

§ 371 Date: Jun. 13, 2017

Related U.S. Application Data
(60) Provisional application No. 62/092,505, filed on Dec. 16, 2014.

(51) Int. Cl. A61B 5/085; A61B 5/087; A61M 16/00; A61M 16/16

(52) U.S. Cl. CPC A61B 5/085; A61M 16/0051; A61B 5/087; A61M 2202/0208; A61M 2202/0007; A61M 2016/0027; A61M 16/16; A61M 2230/42; A61M 2230/46; A61M 2016/0033

(57) ABSTRACT

In a medical ventilator system, a ventilator (10) delivers ventilation to a ventilated patient (12). Sensors (24, 26) acquire airway pressure and air flow data for the ventilated patient. A probabilistic estimator module (40) estimates respiratory parameters of the ventilated patient by fitting a respiration system model (50) to a data set comprising the acquired airway pressure and air flow data using probabilistic analysis, such as Bayesian analysis, in which the respiratory parameters are represented as random variables. A display component (22) displays the estimated respiratory parameters of the ventilated patient along with confidence or uncertainty data comprising or derived from probability density functions for the random variables representing the estimated respiratory parameters.

The following relates to the respiratory therapy arts, respiratory monitoring arts, mechanical ventilation arts, and related arts.

Estimation of respiratory system parameters such as resistance Rᵣₛ and compliance Cᵣₛ is useful for diagnosing respiratory diseases, choosing an appropriate mode of mechanical ventilation (if any), optimizing mechanical ventilator settings for a particular ventilated patient, and so forth.

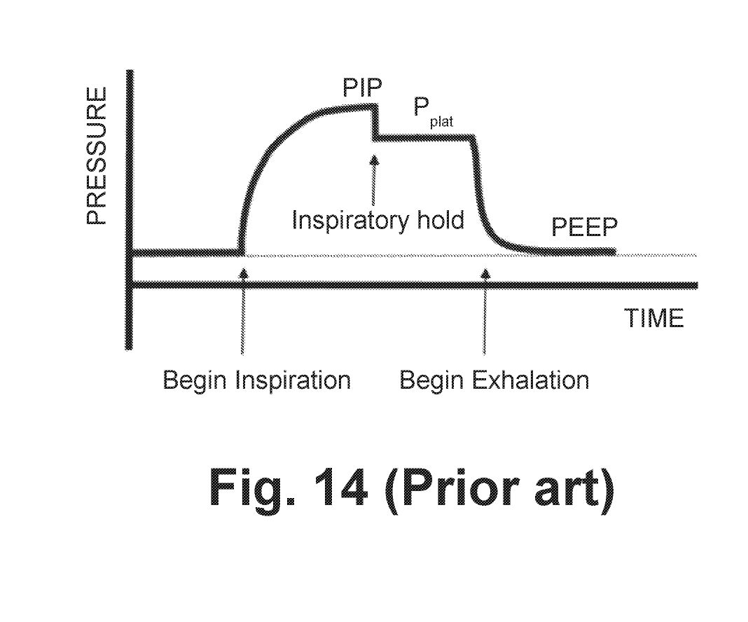

By way of further illustration, a passive mechanically ventilated patient is unable to assist in breathing, and the ventilator performs the entire work of breathing. With reference to FIG. 14, a known technique for assessing respiratory mechanics in a passive mechanically ventilated patient is the End Inspiratory Pause (EIP), also called Flow Interrupter Technique (FIT) or Inspiratory Hold Maneuver. This technique consists of rapidly occluding the circuit through which the patient is breathing under conditions of constant inspiratory flow, while measuring the pressure in the circuit behind the occluding valve. As illustrated in FIG. 14, under conditions of constant inspiratory flow (\dot{V}\dot{V}), airway opening pressure increases from the positive end-expiratory value (PEEP) to peak inspiratory pressure (PIP). When the circuit is occluded, flow is stopped temporarily thus eliminating the resistive pressure component and causing airway opening pressure to drop from PIP to a plateau pressure value (Pₚₗₐₜ). Then the patient is allowed to exhale to set PEEP. The gradient between PIP and Pₚₗₐₜ allows for calculation of airway resistance according to:

$${R}_{rs} = \frac{PIP - {P}_{plat}}{\dot{V}}$$

whereas the value of Pₚₗₐₜ reflects the total elastic recoil pressure and hence allows for calculation of the respiratory system compliance according to:

$${C}_{rs} = \frac{{V}_{t}}{{P}_{plat} - PEEP}$$

where Vₜ is the inhaled tidal volume (computable by integrating air flow \dot{V} over time).

The EIP technique is noninvasive and easy to perform, and commercial ventilators typically have software that automates the EIP procedure and computes resistance and compliance values. However, the EIP technique has certain disadvantages. It interferes with normal operation of the ventilator. Additionally, EIP requires constant inspiratory flow and hence can only be applied in a volume-controlled ventilation (VCV) mode. As a result, EIP is not suitable for continuous monitoring of respiratory mechanics and patient status, and pressure control ventilation (PCV) modes.

The following discloses various improvements.

In accordance with one aspect, a medical ventilator system comprises: a ventilator configured to deliver ventilation to a ventilated patient; an airway pressure sensor configured to acquire airway pressure data for the ventilated patient; an airway airflow sensor configured to acquire airway air flow data for the ventilated patient; a probabilistic estimator module comprising a microprocessor programmed to estimate respiratory parameters of the ventilated patient by fitting a respiration system model to a data set comprising the acquired airway pressure data and the acquired airway air flow data using probabilistic analysis, such as Bayesian analysis, in which the respiratory parameters are represented as random variables; and a display component configured to display the estimated respiratory parameters of the ventilated patient.

In accordance with another aspect, a non-transitory storage medium stores instructions readable and executable by a microprocessor to perform a respiratory parameter estimation method comprising: receiving a data set comprising airway pressure data Pₐₒ(t), airway air flow data \dot{V}(t), and lung volume data V(t) for a ventilated patient receiving ventilation from a mechanical ventilator; and estimating respiratory parameters of the ventilated patient including at least respiratory system resistance Rᵣₛ and respiratory system compliance Cᵣₛ or elastance Eᵣₛ by fitting a respiration system model to the data set using Bayesian analysis in which the respiratory parameters are represented as probability density functions; and causing an estimated respiratory parameter to be displayed on a display device.

In accordance with another aspect, a medical ventilation method comprises: ventilating a patient using a mechanical ventilator; during the ventilating, acquiring a data set comprising airway pressure data Pₐₒ (t) and airway air flow data \dot{V}(t) for the ventilated patient; using a microprocessor, estimating respiratory system resistance Rᵣₛ and respiratory system compliance Cᵣₛ or elastance Eᵣₛ by fitting a respiration system model to the acquired data set using probabilistic analysis in which the respiratory system resistance Rᵣₛ is represented by a probability density function and the respiratory system compliance Cᵣₛ or elastance Eᵣₛ is represented by a probability density function; and displaying the estimated respiratory system resistance Rᵣₛ and respiratory system compliance Cᵣₛ or elastance Eᵣₛ on a display component.

One advantage resides in providing respiratory system resistance Rᵣₛ and compliance Cᵣₛ measurements, which can be applied in substantially any ventilation mode.

Another advantage resides in more accurate estimates of respiratory parameters such as resistance Rᵣₛ and compliance Cᵣₛ, especially for (but not limited to) the case of a passive mechanically ventilated patient.

Another advantage resides in providing estimates of respiratory parameters such as resistance Rᵣₛ and compliance Cᵣₛ, along with estimates of the uncertainties or confidence intervals for those measurements.

Further advantages of the present invention will be appreciated to those of ordinary skill in the art upon reading and understand the following detailed description. It is to be understood that a particular embodiment may achieve none, one, two, some, or all of these advantages.

The invention may take form in various components and arrangements of components, and in various steps and arrangements of steps. The drawings are only for purposes of illustrating the preferred embodiments and are not to be construed as limiting the invention.

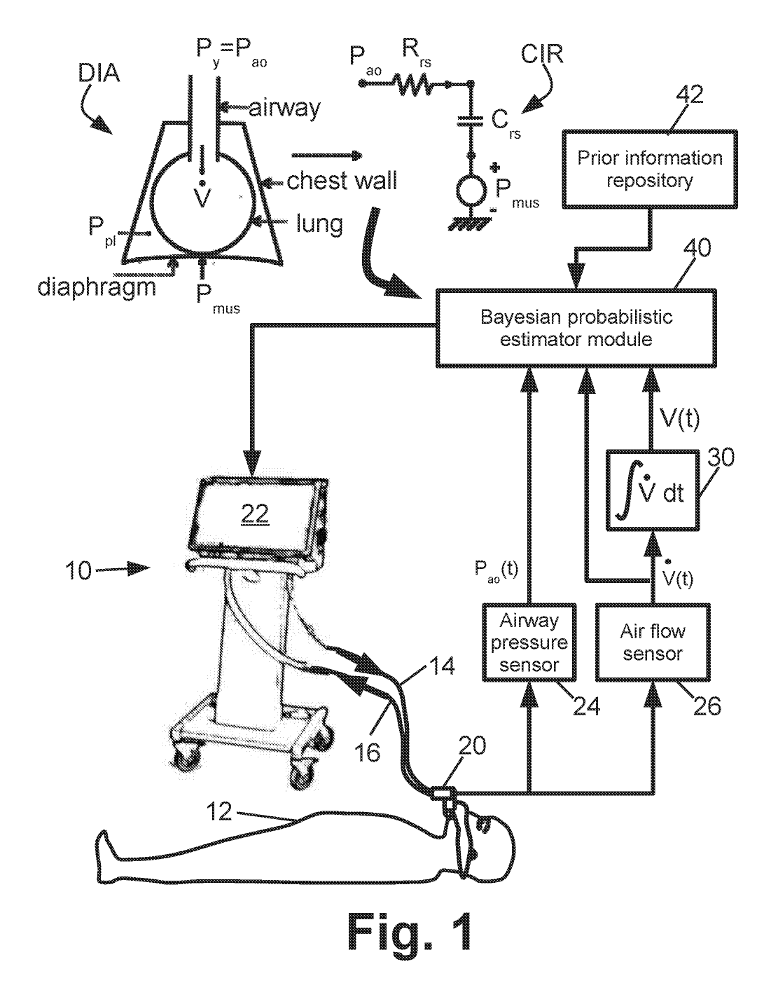

FIG. 1 diagrammatically shows a ventilation system including a probabilistic estimator module for estimating respiratory system resistance Rᵣₛ and compliance Cᵣₛ as disclosed herein.

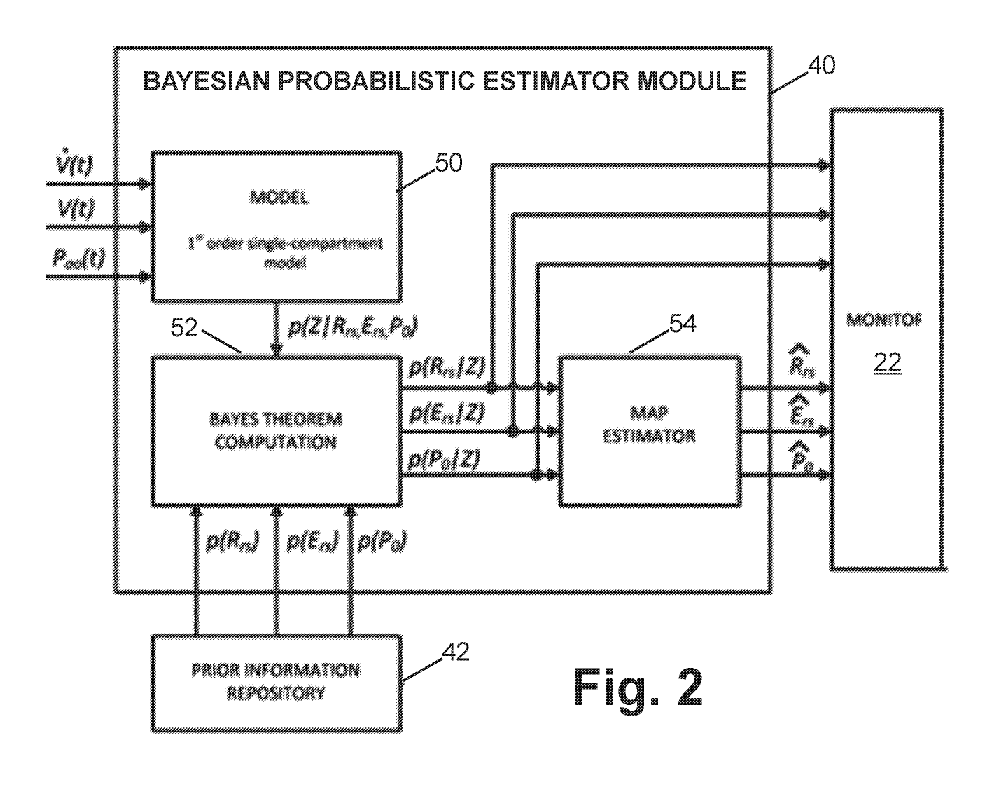

FIG. 2 diagrammatically shows a more detailed representation of the probabilistic estimator module of FIG. 1.

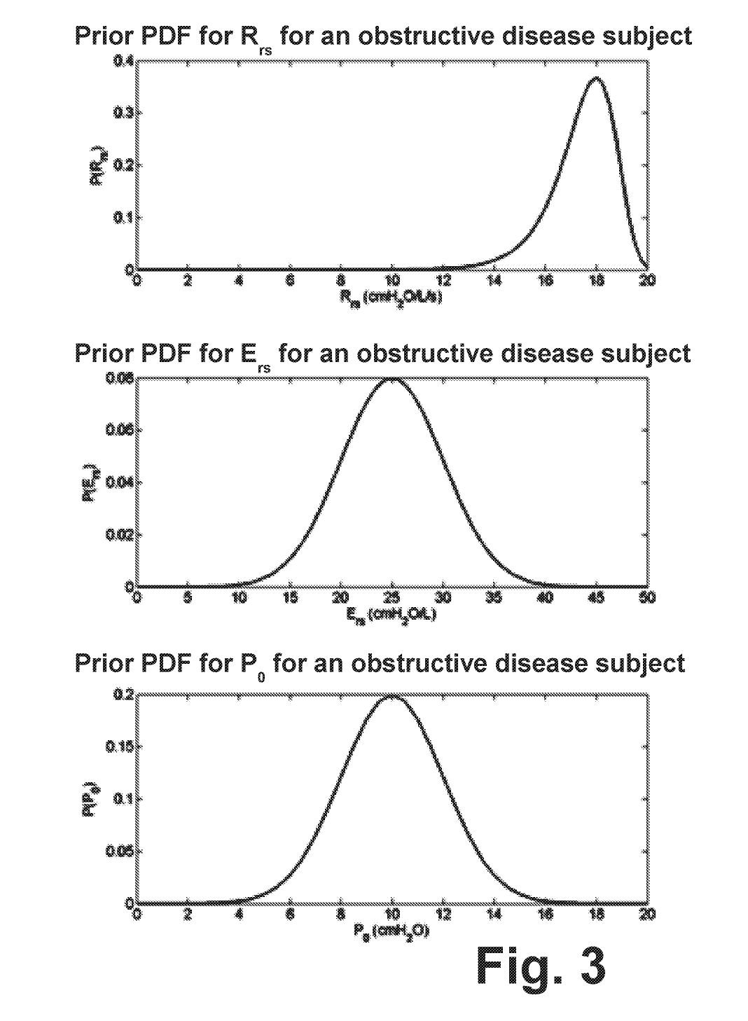

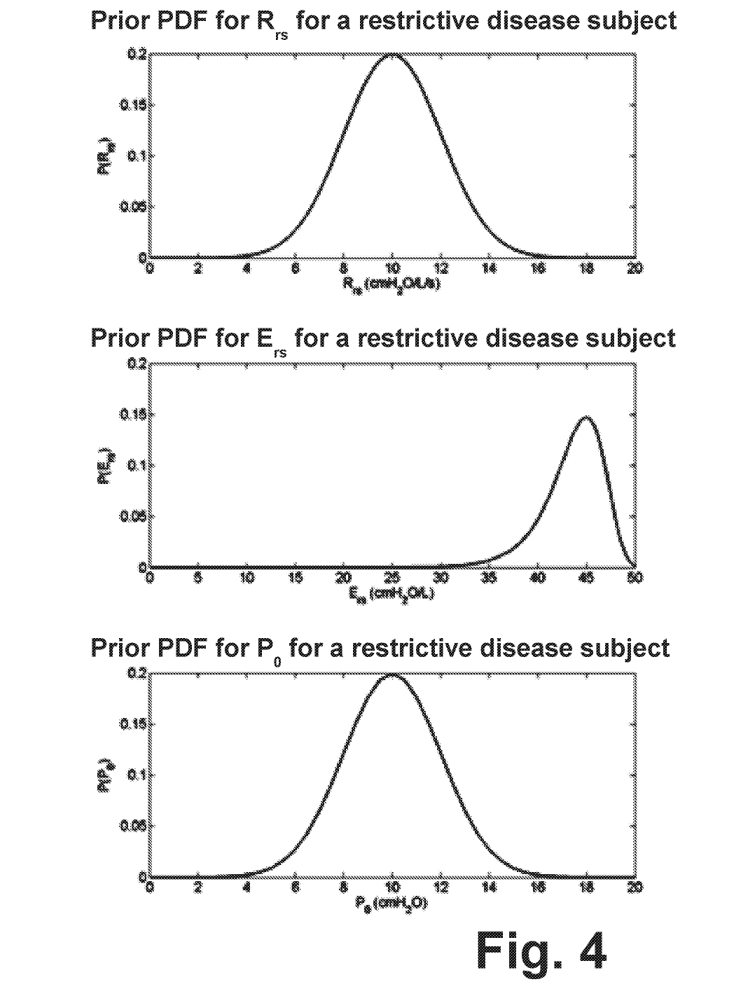

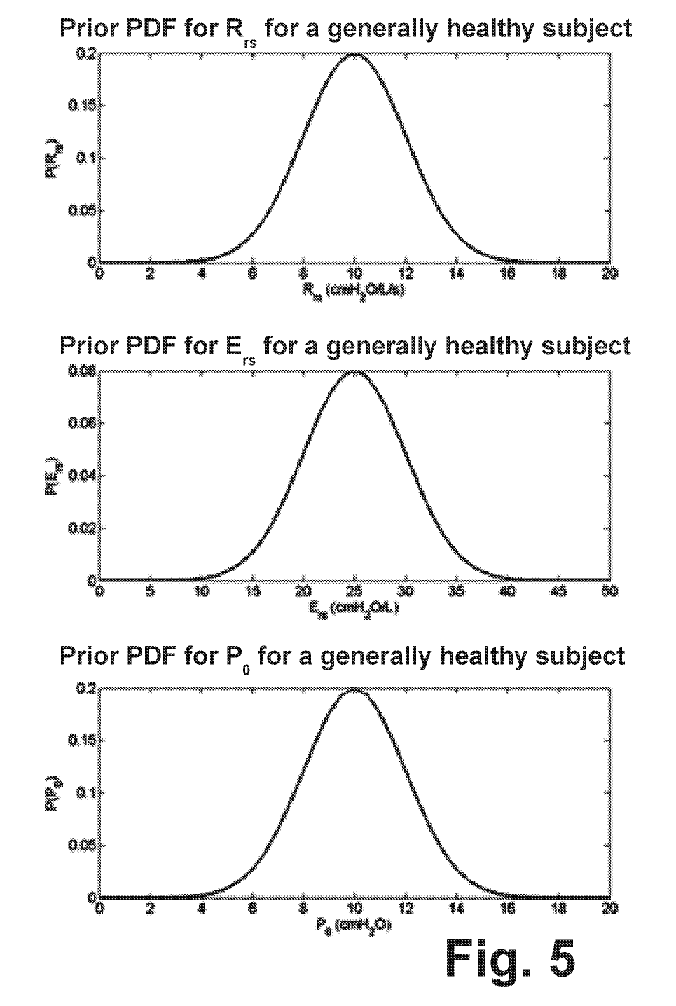

FIGS. 3-5 show a priori probability distribution functions (PDFs) based on prior knowledge for random variables that are evaluated by the probabilistic estimator module of FIG. 1, with: FIG. 3 showing the a priori PDFs for a subject with obstructive disease; FIG. 4 showing the a priori PDFs for a subject with restrictive disease; and FIG. 5 showing the a priori PDFs for a generally healthy subject.

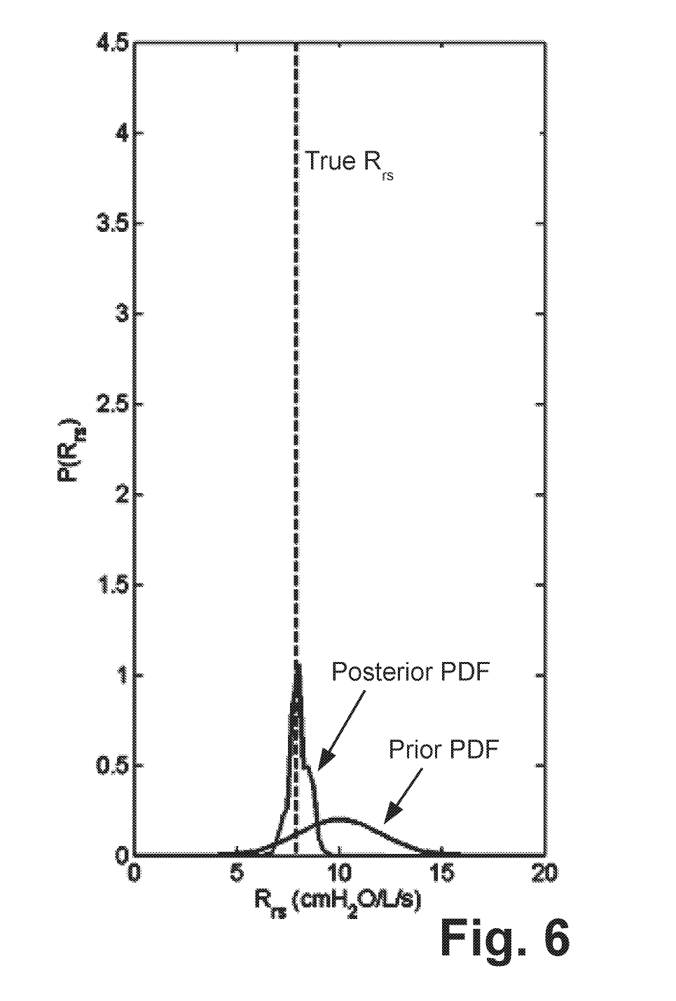

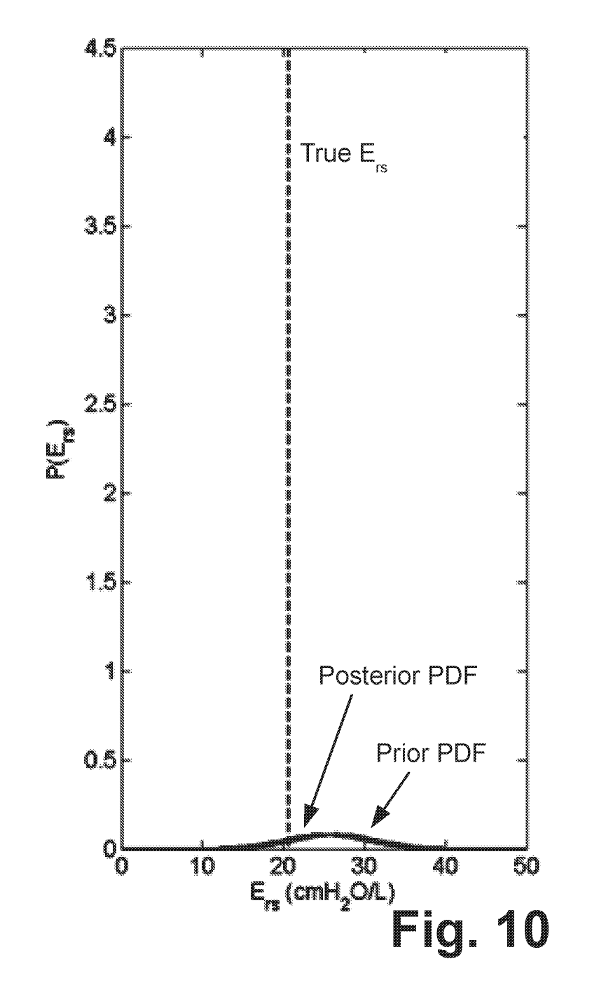

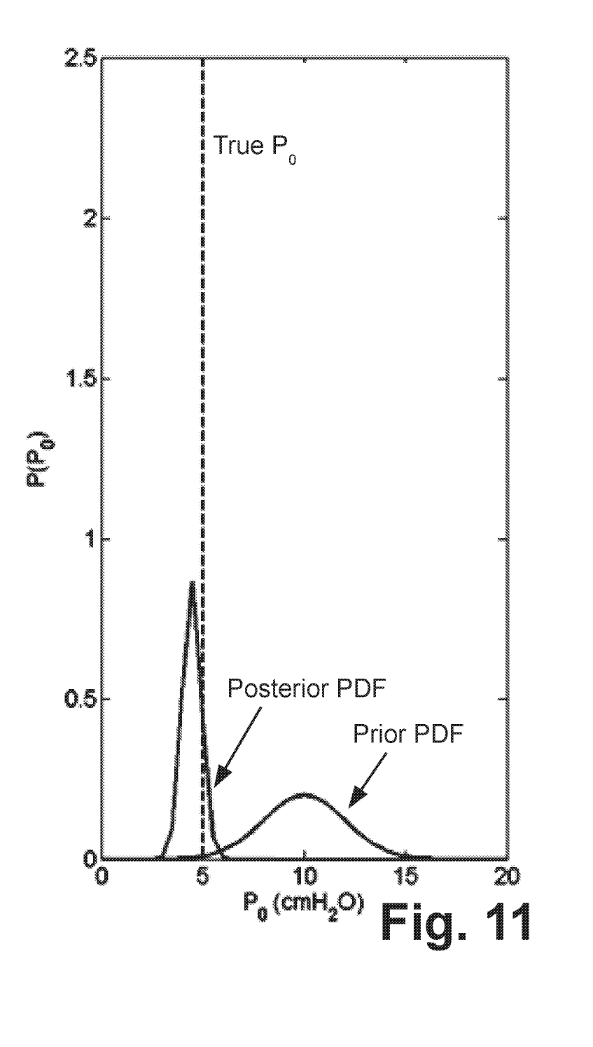

FIGS. 6-11 plot various results for the probabilistic estimator module of FIG. 1 operating on respiratory data acquired from a pig as described herein.

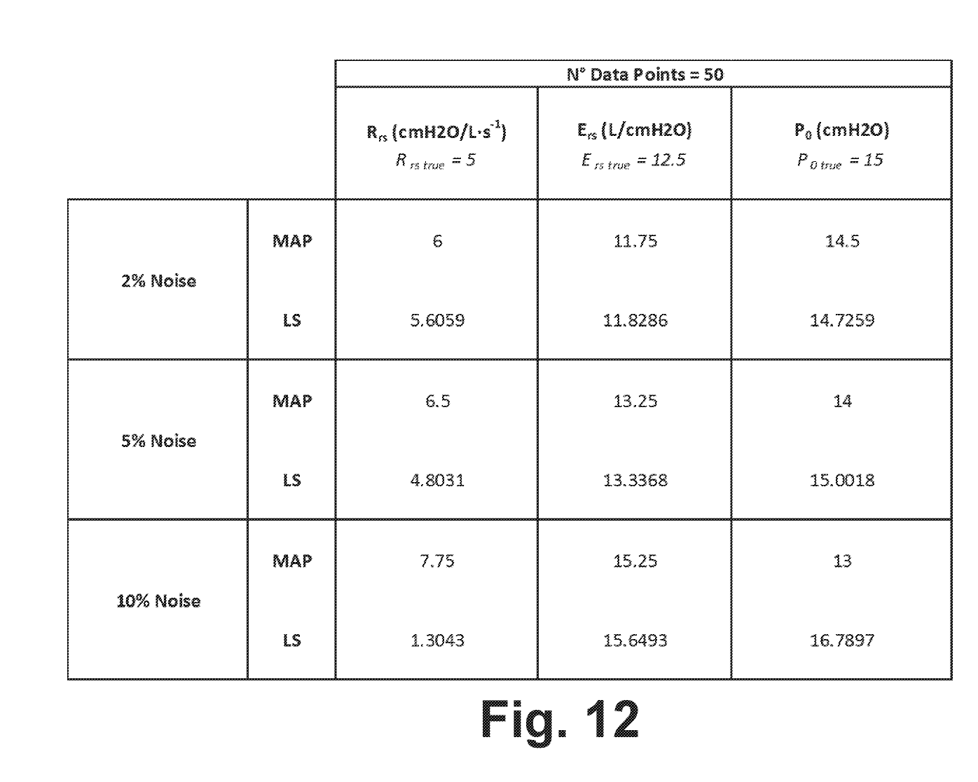

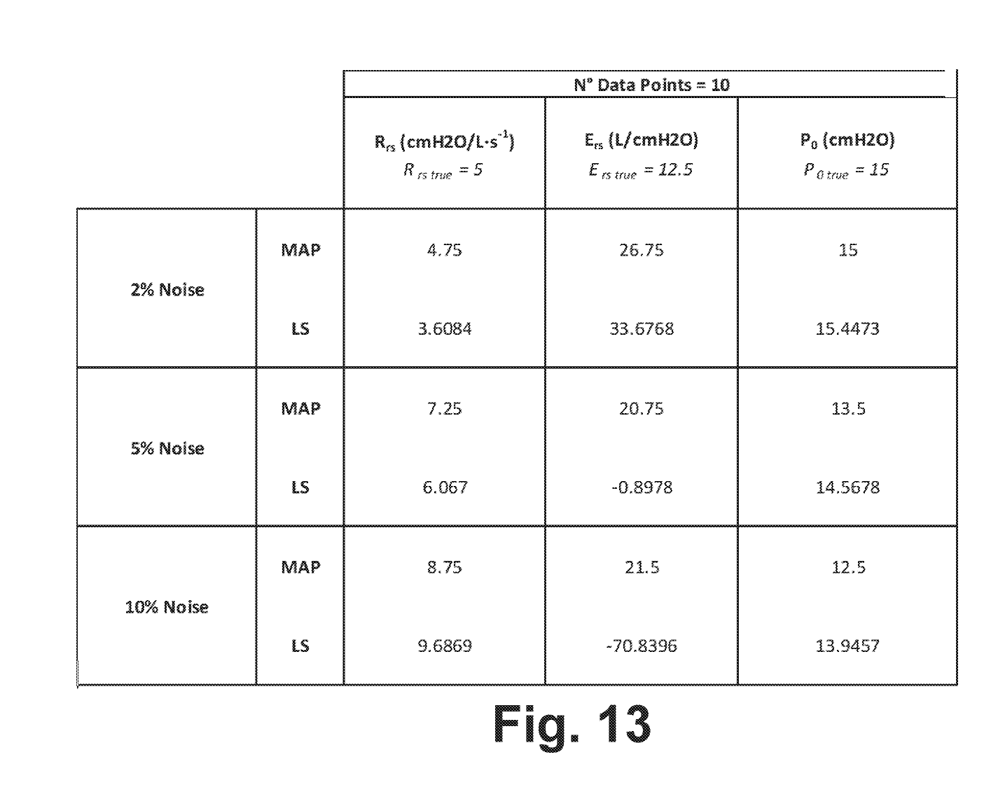

FIGS. 12 and 13 present comparisons of the illustrative Bayesian probabilistic parameter estimation versus least squares estimation, for simulated data as described herein.

FIG. 14 diagrammatically shows operation of the End Inspiratory Pause (EIP) approach for assessing respiratory system resistance Rᵣₛ and compliance Cᵣₛ.

With reference to FIG. 1, a medical ventilator system includes a medical ventilator 10 that delivers air flow at a positive pressure to a patient 12 via an inlet air hose 14. Exhaled air returns to the ventilator 10 via an exhalation air hose 16. A Y-piece 20 of the ventilator system serves to couple air from the discharge end of the inlet air hose 14 to the patient during inhalation and serves to couple exhaled air from the patient into the exhalation air hose 16 during exhalation. Note the Y-piece 20 is sometimes referred to by other nomenclatures, such as a T-piece. Not shown in FIG. 1 are numerous other ancillary components that may be provided depending upon the respiratory therapy being received by the patient 12. Such ancillary components may include, by way of illustration: an oxygen bottle or other medical-grade oxygen source for delivering a controlled level of oxygen to the air flow (usually controlled by the Fraction of Inspired Oxygen (FiO₂) ventilator parameter set by the physician or other medical personnel); a humidifier plumbed into the inlet line 14; a nasogastric tube to provide the patient 12 with nourishment; and so forth. The ventilator 10 includes a user interface including, in the illustrative example, a touch-sensitive display component 22 via which the physician, respiratory specialist, or other medical personnel can configure ventilator operation and monitor measured physiological parameters and operating parameters of the ventilator 10. Additionally or alternatively, the user interface may include physical user input controls (buttons, dials, switches, et cetera), a keyboard, a mouse, audible alarm device(s), indicator light(s), or so forth.

With continuing reference to FIG. 1, the patient 12 is monitored by various physiological parameter sensors. In particular, FIG. 1 illustrates two such sensors: an airway pressure sensor 24 that measures air flow V̅(t) to or from the patient (usually measured at the Y-piece 20), and an air flow sensor 26 that measures pressure at the coupling to the patient (usually also measured at the Y-piece 20). This pressure is denoted herein as P_{y} (t) (since it is usually measured at the Y-piece 20) or Pₐₒ (t) (the airway opening pressure). Other physiological parameters are conventionally monitored by suitable sensors, such as heart rate, respiratory rate, blood pressure, blood oxygenation (e.g. SpO₂), respiratory gases composition (e.g. a capnograph measuring CO₂ in respiratory gases), and so forth. Other physiological parameters may be derived from directly measured physiological parameters—by way of illustration, a lung volume determination component 30 computes net air flow into the patient 12 by integration of the air flow \dot{V}(t) over the salient time period (e.g. one breath intake).

An alternative to the EIP maneuver for measuring respiratory system resistance Rᵣₛ and compliance Cᵣₛ is to perform a Least Squares (LS) fit of a mathematical model of a measured respiratory waveform, e.g. the airway pressure waveform Pₐₒ (t) and/or the airway flow waveform \dot{V}(t) obtained noninvasively at the opening of the patient airway. A suitable model is a first-order linear single-compartment model that describes the respiratory system as an elastic compartment served by a single resistive pathway. FIG. 1 illustrates a schematic diagram DIA of the first-order linear single-compartment model, as well as an electrical analog circuit CIR. In the diagram DIA, the pressure Pₚₗ denotes the pressure of the compartment representing the pleural space. The governing equation of the first-order linear single-compartment model, also known as the equation of motion of the respiratory system, can be written as:

Pₐₒ(t)=Rᵣₛ·\dot{V}(t)+Eᵣₛ·V(t)+Pₘᵤₛ(t)+P₀ (1)

where Pₐₒ is the airway opening pressure, \dot{V}\dot{V} is the air flow, VV is the lung volume above functional residual capacity (FRC), Pₘᵤₛ is the pressure generated by the patient respiratory muscles (driving source), Rᵣₛ is the respiratory system resistance, Eᵣₛ is the respiratory system elastance (inverse of the compliance Cᵣₛ, that is,

$${E}_{rs} = \frac{1}{{C}_{rs}} ) ,$$

and P₀ is a constant term added to account for the pressure that remains in the lungs at the end of expiration. In a passive patient who is not breathing spontaneously, the term Pₘᵤₛ in Equation (1) can be removed:

Pₐₒ(t)=Rᵣₛ·\dot{V}(t)+Eᵣₛ·V(t)+P₀+w(t) (1a)

where an extra term w(t)w(t) has been included in Equation (1a) in order to account for the presence of measurement error and model error.

Equation (1a) is applied to a time series of samples at times t₁, . . . , t_{N} (that is, a time sequence of N samples indexed 1, . . . , N) yields the following matrix equation:

$$Z ≡ [ \begin{matrix}{P}_{ao} ( {t}_{1} ) \\ {P}_{ao} ( {t}_{2} ) \\ ⋮ \\ {P}_{ao} ( {t}_{N} )\end{matrix} ] = [ \begin{matrix}\dot{V} ( {t}_{1} ) & V ( {t}_{1} ) & 1 \\ \dot{V} ( {t}_{2} ) & V ( {t}_{2} ) & 1 \\ & ⋮ & \\ \dot{V} ( {t}_{N} ) & V ( {t}_{N} ) & 1\end{matrix} ] \cdot [ \begin{matrix}{R}_{rs} \\ {E}_{rs} \\ {P}_{0}\end{matrix} ] + [ \begin{matrix}w ( {t}_{1} ) \\ w ( {t}_{2} ) \\ ⋮ \\ w ( {t}_{N} )\end{matrix} ] = H \cdot θ + W \qquad ( 2 )$$

Matrix Equation 2 represents a tractable linear regression problem, where H is the matrix containing the input variables, Z is the output vector, θ θ is the parameter vector containing the unknown parameters (Rᵣₛ, Eᵣₛ and P₀), and N is the number of samples. Hence, in the case of fully passive patients, an estimate of the parameter vector \hat{θ}\hat{θ} (containing the estimated resistance and compliance) can be obtained via the classical Least Squares (LS) method:

\hat{θ}=(H^{T}H)⁻¹H^{T}Z (2a)

provided that airway pressure Pₐₒ and flow \dot{V}(t) at the patient's airway entrance (e.g. mouth or tracheostomy tube) are measured. The lung volume V is obtained by numerical integration of the flow signal \dot{V}(t) performed by the lung volume determination component 30.

The least squares (LS) technique using a first-order single-compartment model is a non-invasive alternative to the EIP maneuver. The LS technique advantageously does not interfere with the normal operation of the mechanical ventilator 10, and allows for continuous monitoring of respiratory mechanics during normal ventilation.

However, least squares fitting is an iterative process that is sensitive to factors such as the initial values used to initiate the iterating, noise in the data, the number of iterations, the stopping criteria employed to terminate the iterating, possible settling upon a local minimum, and so forth. Least squares fitting typically does not leverage a priori knowledge about Rᵣₛ and Cᵣₛ, even though such knowledge may be available from population studies and/or domain expert (clinicians or data bases). For instance, given statistics for past patients belonging to a particular class of patients, it is possible to identify certain values of Rᵣₛ and Cᵣₛ as being more likely than others, based on previous studies or physiological knowledge. At most, the LS optimization may use such prior knowledge to choose initial values for the parameters to be fit, but this leverages only a part of the available prior information. The LS technique can also become inaccurate when significant noise is present in the measurements or few data samples are used. In addition, LS techniques provide estimated parameter values, but generally do not provide a confidence or uncertainty metric for these estimated values.

With continuing reference to FIG. 1, the medical ventilator systems disclosed herein employ probabilistic estimation, such as via an illustrative Bayesian probabilistic estimator module 40, or using a Markovian process, in order to fit a model of the respiratory waveform, such as the illustrative first-order linear single-compartment model represented by Equations (1) and (1a). In such a process, the parameters of interest, e.g. resistance Rᵣₛ, compliance Cᵣₛ (or elastance Eᵣₛ), as well as other fitted parameters such as P₀, are represented as random variables described by probability density functions (PDF's). Advantageously, prior information from a repository 42 can be leveraged as a priori PDFs in the probabilistic estimation process. Such an a priori PDF based on prior information advantageously captures not just the mean or average of the prior information, but also its breadth, variance or the like. The output of the probabilistic estimation process is not a single value, but rather an optimized PDF. The peak, average, mean, or the like of this PDF then provides the estimated value (similar to what is output by a LS algorithm), but the width or other metric characterizing the spatial extent of the PDF additionally provides a measure of the confidence or uncertainty of the estimated value. In some embodiments, the PDF itself may be plotted to provide a visual depiction of the confidence or uncertainty. The probabilistic estimation process operates to (usually, when the patient 12 is stable) narrow the width or extent of the PDF over time as more data becomes available. The leveraging of prior information in the probabilistic estimation process also makes it more robust to noise as compared with LS approaches. Hence, it provides more accurate and precise estimates even when high noise is present in the measurements or too few data samples are used/collected.

The disclosed probabilistic estimation approaches estimate respiratory system resistance, Rᵣₛ, and compliance, Cᵣₛ (or elastance Eᵣₛ) using the input data airway pressure Pₐₒ(t), airway flow \dot{V}(t) and lung volume V(t). In FIG. 1, the physiological parameters Pₐₒ(t) and \dot{V}(t) are measured non-invasively at the airway opening of the patient (such as at the Y-piece 20) by the sensors 24, 26. Physiological parameter V(t) is suitably obtained by numerical integration of \dot{V}(t) using the lung volume determination component 30. These serve as inputs to the illustrative Bayesian probabilistic estimator module 40, which outputs both numerical values for the estimated parameters and posterior probability density functions (PDFs) of the estimated parameters providing confidence/uncertainty.

With continuing reference to FIG. 1 and with further reference to FIG. 2 which depicts a more detailed block diagram of the probabilistic estimator module 40, the illustrative Bayesian probabilistic estimator module 40 employs the first-order single-compartment model of the respiratory system shown in FIG. 1 schematic diagram DIA and electrical analog circuit CIR to relate the measurement vector Z to the parameter vector θ in accordance with Equation (2). In FIG. 2, the first-order single-compartment model is denoted by reference number 50. In the probabilistic estimation framework, the unknown parameter vector θ is treated as a random variable. The a priori knowledge about the parameters contained in the repository 42 is summarized via a probability density function p(θ) (prior PDF or a priori PDF). This PDF is updated as new measurements become available (each new measurement adds a row to the matrix Equation (2), and a posterior parameter PDF p(θ|Z) is computed by applying Bayes' theorem:

$$p ( θ \vert Z ) = \frac{p ( Z \vert θ ) \cdot p ( θ )}{p ( Z )} \qquad ( 3 )$$

where p(Z|θ) is the conditional PDF of the measurements Z given the parameters θ, also called “likelihood” function, and p(Z) is the PDF of the measurements Z. In FIG. 2, a block 52 denotes the Bayes theorem computation. With p (θ|Z) computed, an estimate of the parameter vector B is obtained according to the Maximum a Posteriori Probability (MAP) estimator as the mode of the posterior PDF p(θ|Z):

$${\hat{θ}}_{MAP} = {argmax}_{θ} \{ p ( θ \vert Z ) \} \qquad ( 4 )$$

In FIG. 2, the MAP estimator is denoted by a block 54. The estimated parameter vector θ is suitably decomposed into its constituents, i.e. an estimated respiratory system resistance \hat{R}ᵣₛ, an estimated respiratory system elastance \hat{R}ᵣₛ (or, equivalently, an estimated respiratory system compliance Ĉᵣₛ=1/Êᵣₛ), and an estimated \hat{P}₀. These values are suitably displayed on the display component 22 of the mechanical ventilator 10, or on another display component (e.g. on a desktop computer running the probabilistic estimator, or so forth).

Additional notation used in FIG. 2 includes the following: Pₐₒ (t) denotes the airway pressure signal; \dot{V}(t) denotes the airflow signal; V(t) denotes the lung volume signal; p(Rᵣₛ) denotes the prior PDF for the respiratory system resistance; p(Eᵣₛ) denotes the prior PDF for the respiratory system elastance; p(P₀) denotes the prior PDF for the baseline pressure P₀; p(Z|Rᵣₛ, Eᵣₛ, P₀) denotes the likelihood function; p(Rᵣₛ|Z) denotes the posterior PDF of the respiratory system resistance; p(P₀|Z) denotes the posterior PDF of the baseline pressure P₀; Rᵣₛ denotes the estimated respiratory system resistance; E denotes the estimated respiratory system elastance; and P₀ denotes the estimated baseline pressure P₀.

In order to compute the posterior PDF p(θ|Z), as shown in Equation (3), the following operations are performed: determining the prior probability density function p(θ); computing of the likelihood function p(Z|θ); and computing the posterior probability density function p(θ|Z). Each of these operations are described in succession next.

The prior probability density function p(θ) is suitably determined from prior knowledge. This entails defining the individual prior PDF of the parameters to be estimated, which for the first-order linear single-compartment model include resistance Rᵣₛ, elastance Eᵣₛ, and the additional fitting parameter P₀. In order to create the prior distributions, the parameters Rᵣₛ, Eᵣₛ and P₀ are given a range of possible values and this range is discretized. Then, within these ranges, the parameters are assumed to be distributed according to a chosen probability density function (prior PDF). The choice of the prior PDF depends on population studies and clinicians knowledge.

With reference to FIGS. 3-5, determination of the prior PDFs is described for three patient classes: a subject with obstructive disease (FIG. 3): a patient with restrictive disease (FIG. 4); and a generally healthy subject (FIG. 5). If a diagnosis of obstructive disease has been made on the patient, then it is reasonable to assume that high values of Rᵣₛ are most likely to occur, hence the prior PDFs shown in FIG. 3 are suitably chosen. On the other hand, if a diagnosis of restrictive disease has been made, then it is reasonable to assume that higher values of elastance Eᵣₛ are most likely to occur, hence the prior PDFs of FIG. 4 are suitably chosen. Finally, if a patient is considered healthy, then Gaussian PDFs shown in FIG. 5 which are centered around median values of the corresponding parameter ranges can be chosen. If no prior knowledge is available, then the prior PDFs can be assumed to be uniform (within some minimum-to-maximum range) to indicate that all possible parameter values are equally probable.

With the individual prior PDFs defined, and under the assumption that the parameters are independent, the joint prior PDF p(θ) is computed as the product of the individual priors:

P(θ)=p(Rᵣₛ)·p(Eᵣₛ)·p(P₀) (5)

where p(Rᵣₛ) is the prior PDF for the resistance Rᵣₛ, and p(Eᵣₛ) is the prior PDF for the compliance Eᵣₛ, and p(P₀) is the prior PDF for the additional parameter P₀.

The next operation is computing of the likelihood function p(Z|θ). This can be achieved by evaluating the first-order single-compartment model 50 of the respiratory system for the possible values of the parameter vector θ and taking into account the noise term W. Particularly, if W is assumed to be white Gaussian noise with zero mean and covariance matrix C_{W}=σ_{w}²·I_{N} (where I_{N} is the N×N identity matrix), then the random vector Z|θ is a multivariate Gaussian variable with mean equal to H·θ and covariance matrix equal to C_{W}. Hence, the likelihood function can be computed as:

$$p ( Z \vert θ ) = \frac{1}{{[ {( 2 π )}^{N} det ( {C}_{w} ) ]}^{1 / 2 }} \cdot {e}^{- \frac{1}{2} {( Z - H θ )}^{T} \cdot {C}_{w}^{- 1} \cdot ( Z - H θ )} \qquad ( 6 )$$

The third operation is computing the posterior probability density function p(θ|Z). This entails executing the product and division operations of Bayes' theorem (Equation (3)) in order to obtain the posterior PDF p(θ|Z). Computation of the product p(Z|θ)·p(θ) is straightforward. Division by p(Z) requires the term p(Z) to be computed first. To this end, it is recognized that the term p(Z|θ)·p(θ) represents the joint PDF of the random vectors Z and θ:

p(z,θ)=p(z|θ)·p(θ) (7)

Hence, in order to compute p(Z), the joint p.d.f. p(Z, θ) that has just been computed is marginalized according to:

p(Z)=∫_{θ}p(Z,θ)dθ=∫_{θ}p(Z|θ)·p(θ)dθ (8)

Finally, in order to compute the individual posterior PDFs p(Rᵣₛ|Z), p(Eᵣₛ|Z) and p(P₀|Z), the joint PDF p(θ|Z) is marginalized according to:

P(Rᵣₛ|Z)=∫_{E}_{ᵣₛ}(∫_{P}_{₀}p(θ|Z)dP₀)dEᵣₛ

p(Eᵣₛ|Z)=∫_{R}_{ᵣₛ}(∫_{P}_{₀}p(θ|Z)dP₀)dRᵣₛ

p(P₀|Z)=∫_{E}_{ᵣₛ}((∫_{R}_{ᵣₛ}p(θ|Z)dRᵣₛ)dEᵣₛ (9)

The disclosed approaches for estimating respiratory parameters using probabilistic estimation provide a non-invasive way to assess respiratory mechanics, i.e. respiratory system resistance Rᵣₛ and compliance Cᵣₛ, in passive patients continuously and in real time. Not only do these approaches provide values for the estimated parameters, but also PDFs that offer visually interpretable information to bedside clinicians or attending clinicians in the critical care setting. These PDFs can be plotted on the display component 22 of the ventilator 10, or on a patient monitor, mobile device, or other display-capable device. The PDFs indicate both the most likely value of the parameter under exam (Rᵣₛ or Cᵣₛ) and the uncertainty associated with the estimates.

With continuing reference to FIGS. 1 and 2, a more detailed embodiment of the Bayesian probabilistic estimator module 40 is described. The patient 12 is connected to the mechanical ventilator 10 either invasively, e.g. using a tracheostomy tube, or non-invasively, e.g. via an tracheal tube or catheter. Airway pressure (Pₐₒ) and flow (\dot{V}) are measured at the patient's mouth via the sensors 24, 26. Lung volume (V) is obtained from the flow measurements \dot{V} via numerical integration performed by the component 30. The measurements Pₐₒ (t), \dot{V}(t), and V(t) are fed in real-time to the probabilistic estimator module 40. To perform the Bayesian probabilistic parameter estimation, the mathematical model 50 of the respiratory system is applied, e.g. the first-order single-compartment model diagrammatically shown in the upper inset of FIG. 1. For the first-order single-compartment model 50, this entails evaluating matrix Equation (2) for all the possible parameter values to construct the likelihood function p(Z|Rᵣₛ, Eᵣₛ, P₀). The Bayes theorem computing component 52 receives the prior PDFs p(Rᵣₛ), p(Eᵣₛ) and p(P₀), e.g. from the past patients data repository 42, and combines them with the likelihood function p(Z|Rᵣₛ,Eᵣₛ,P₀), and computes the posterior parameter PDFs p(Rᵣₛ|Z), p(Eᵣₛ|Z) and P(P₀|Z). The maximum a-posteriori probability (MAP) estimator 54 computes the maximum of the posterior PDF \hat{θ} which is decomposed to yield the estimates of the parameters \hat{R}ᵣₛ, Êᵣₛ and \hat{P}₀.

The prior information repository 42 is used to generate the prior PDFs based on clinician's inputs, such as patient's diagnosis, demographic information, health history, patient's class etc. Furthermore, the posterior PDF and the estimated parameter values are displayed on a monitor, e.g. the ventilator display component 22, a patient monitor or a mobile device for remote monitoring.

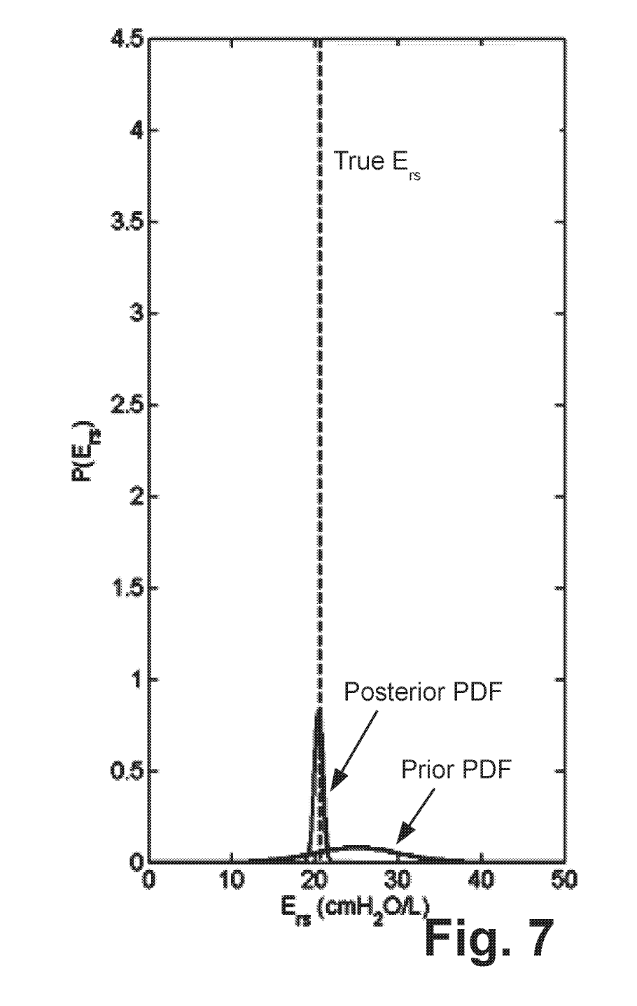

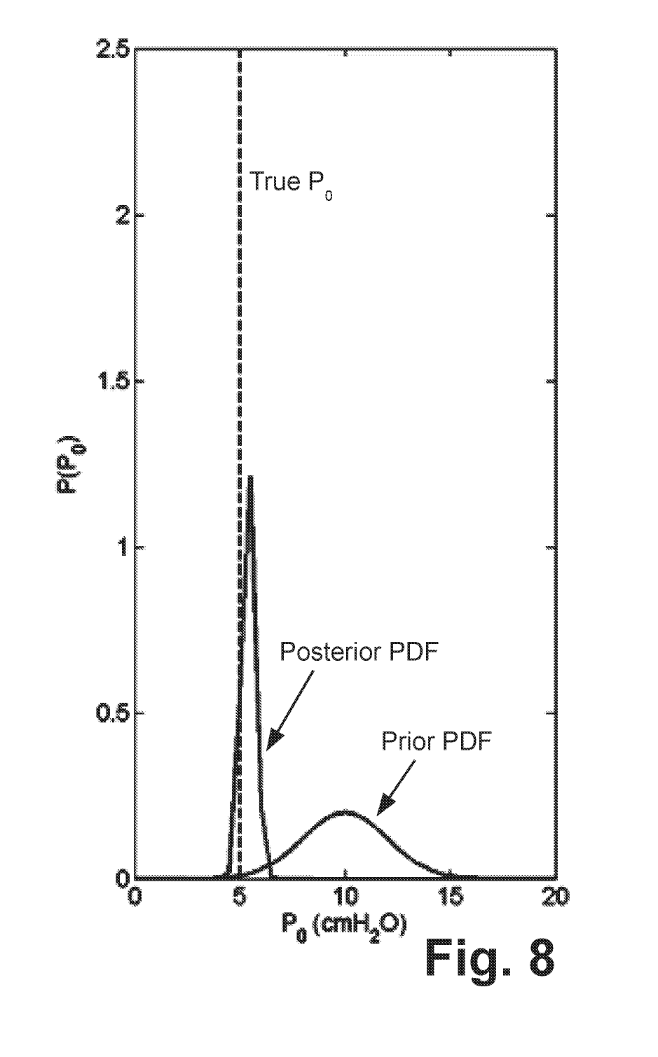

With reference to FIGS. 6-8, an example of results provided by the disclosed Bayesian probabilistic parameter estimator 40 is described. The results have been obtained using experimental data taken from pig. Particularly, 100 samples of pressure (Pₐₒ), flow (\dot{V}) and volume (V) measurements have been used to compute the posterior PDF of Rᵣₛ, Eᵣₛ and P₀ starting from their prior PDFs. In this example, the prior PDFs were chosen to be Gaussian, assuming that the “patient” (i.e. the pig) is healthy and no diagnosis of respiratory disease is made. The indicated “true” values for the parameters to be estimated (Rᵣₛ, Eᵣₛ and P₀) were obtained via the EIP technique and are indicated in FIGS. 6-8, along with indicated plots of the Gaussian prior PDF and the posterior PDF. FIG. 6 plots the results for resistance (Rᵣₛ), while FIG. 7 plots the results for elastance (Eᵣₛ) and FIG. 8 plots the results for parameter P₀.

FIGS. 6-8 illustrate that in this experiment the Bayes probabilistic parameter estimation provided posterior PDFs that are centered on the corresponding true (i.e. EIP-measured) parameter values, indicating that the Bayes probabilistic parameter estimation provides results in agreement with the gold-standard EIP method without interfering with the ventilator. The posterior PDFs are also narrowed substantially compared with the prior PDFs, indicating high levels of confidence of the estimated parameters. The confidence of each parameter is readily discerned by visual review of the plotted posterior PDFs, and in some contemplated embodiments the posterior PDFs are contemplated to be plotted on the display component 22 of the ventilator 10 (or on another display device).

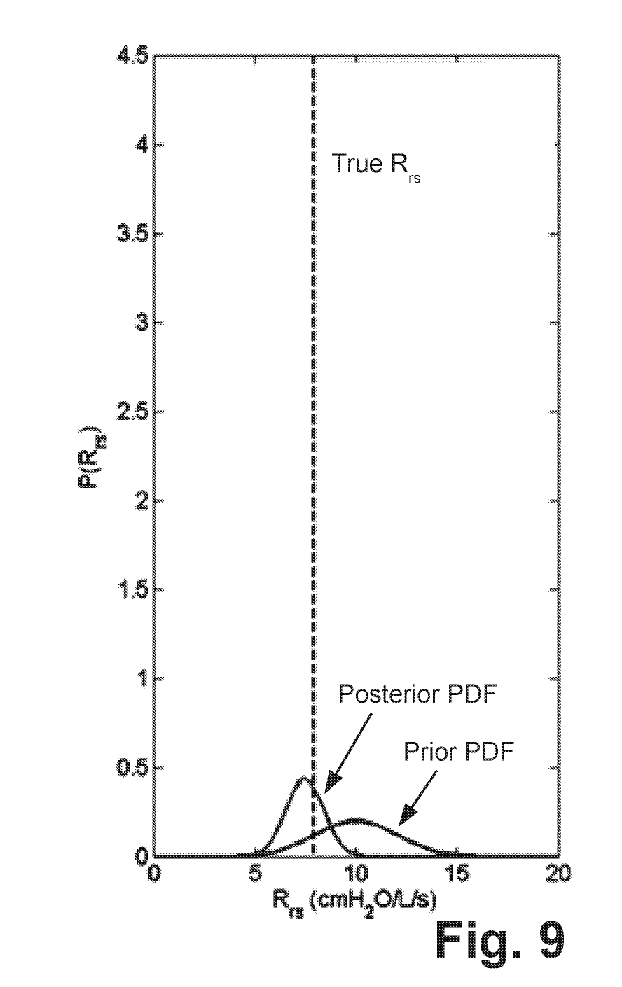

FIGS. 9-11 illustrate results corresponding to respective FIGS. 6-8, but obtained by considering a reduced number of data samples (10 data samples in FIGS. 9-11 as compared with 100 data samples in FIGS. 6-8). Due to the reduced amount of data, the confidence level of the estimated parameters decreases (as seen by wider posterior PDF peaks) because less information is available. This can be easily recognized by the user if the posterior PDFs are plotted on the display component 22.

Real-time patient monitoring can be implemented using the disclosed approach in various ways. In one approach, the Bayesian probabilistic parameter estimator 40 is applied for each successive group or window of N measurements, in a sliding window approach. The Bayesian analysis in the first window uses prior PDFs generated from the past patient data in the repository 42. Thereafter, for each next window of N points, the posterior PDFs generated by the Bayesian analysis of the immediately previous window in time are suitably used as prior PDFs for the next window. In this way the system provides real-time values for the estimated parameters with a temporal resolution on the order of the window size. For example, if N=100 and samples are acquired every 0.6 sec, then the window has duration 60 sec (1 minute). Use of the posterior PDFs of the last window as the prior PDFs of the next window is premised on the expectation that Rᵣₛ, Eᵣₛ, and P₀ are continuous and slowly varying (or constant) in time. It is contemplated for successive windows to overlap in time to provide smoother updating. In the overlap limit of window size N and overlap N−1, the parameters are updated each time a new sample is measured. Optionally, the user can set the window size, e.g. using a slider on the display—increasing the window size increases N and hence provides narrower posterior PDFs (compare FIGS. 6-8 with N=100 compared with FIGS. 9-11 with N=10), but at the cost of lower temporal resolution.

If the parameter distributions p(Rᵣₛ), p(Eᵣₛ) and p(P₀) are not independent, then it may be advantageous to preserve the full joint distribution across successive time windows. In other words, rather than using the individual PDFs p(Rᵣₛ), p(Eᵣₛ) and p(P₀) as priors in performing the Bayesian analysis for the next time window, it may be preferable to use the joint posterior distribution as the prior for the next time window. See Equation (5) and related text which discusses the joint prior p(θ). In this case, the marginal probabilities (that is, the individual posterior PDFs p(Rᵣₛ|Z), p(Eᵣₛ|Z) and p(P₀|Z) marginalized in accord with Equation (9)) are generated only for the display.

With reference to FIGS. 12 and 13, the Bayesian probabilistic parameter estimator module 40 provides robust parameter estimation. To demonstrate, performance of the Bayesian probabilistic parameter estimation is compared with least squares (LS) estimation in the tables presented in FIGS. 12 and 13 for data-poor conditions, i.e. when the noise level is high (2%, 5%, or 10% noise in the examples of FIGS. 12-13) and the number of data samples used in the estimation process is low (N=50 for the table of FIG. 12, and N=10 for the table of FIG. 13). In the tables of FIGS. 12-13, the label “MAP” indicates Bayesian probabilistic parameter estimation, while the label “LS” indicates least squares estimation. The improved robustness of the Bayesian probabilistic approach is attributable to the additional use of prior knowledge about the parameters. The results presented in the tables of FIGS. 12-13 were obtained via simulation studies, in which nominal values for the parameters were fixed and simulated airway pressure signals were generated by solving Equation (2) using these nominal parameter values and the experimental flow and volume data from the same pig experiment described with reference to FIGS. 6-11. A noise term w(t) has been added to the simulated airway pressure signal, according to Equation (2). Different noise levels have been investigated. Particularly, the noise has been assumed to be white Gaussian with zero mean and standard deviation equal to 2%, 5% or 10% of the dynamic range of the pressure signal, indicating low, medium and high noise conditions, respectively. As shown in the tables of FIGS. 12 and 13, when the noise level is high and the number of data samples is reduced (N=50 in FIG. 12, or N=10 in FIG. 13), the LS technique provided unrealistic parameter values (sometime even negative), whereas the Bayesian probabilistic parameter estimation provided values that are in a physiological range and relatively close to their nominal values.

The illustrative Bayesian probabilistic parameter estimation is an example, and numerous variants are contemplated. For example, the probabilistic parameter estimation can use a probabilistic estimation process other than Bayesian estimation, such as Markovian estimation. The probabilistic parameter estimation should receive as inputs the data within the window and the a priori PDFs, and should output posterior PDFs.

In other contemplated variants, the first-order single compartment model 50 can be replaced by a different respiration system model, such as one in which the respiratory system resistance is replaced by a flow-dependent resistance, that is, Rᵣₛ=R₀+R₁·|\dot{V}(t)|. In this case, the parameters estimated by the Bayesian probabilistic parameter estimation include the resistance parameters R₀ and R₁. Similarly, the elastance can be replaced by a volume-dependent elastance, that is, Eᵣₛ=E₀+E₁V(t) where the parameters to be estimated are E₀ and E₁.

In another contemplated variation, the estimator block 54 may use a different criterion beside the illustrative Maximum a Posteriory Probability (MAP) criterion. With the posterior PDFs p(Rᵣₛ|Z), p(Eᵣₛ|Z) and p(P₀|Z) computed, other point estimators can be used to choose the estimated parameter values based on their corresponding posterior PDFs For instance, the Minimum Mean Square Error estimator that will select the estimates as the mean of the posterior p.d.f. could be used:

θ_{MMSE}=E{θ|Z} (10)

In further contemplated variations, the output of the Bayesian probabilistic parameter estimation can be variously displayed. For example, the actual PDFs may or may not be displayed—if the are not displayed, then it is contemplated to display a metric measuring the PDF width, such displaying a confidence interval numeric values as a half-width-at-half-maximum (HWHM) of the posterior PDF peak. The display could, for example, be formatted as “XXX±YYY” where “XXX” is the estimated value (e.g. \hat{R}ᵣₛ) and “YYY” is the HWHM of the posterior PDF representing Rᵣₛ.

With returning reference to FIG. 1, the data processing components 30, 40 are suitably implemented as a microprocessor programmed by firmware or software to perform the disclosed operations. In some embodiments, the microprocessor is integral to the mechanical ventilator 10, so that the parameter estimation is performed by the ventilator 10. In other embodiments the microprocessor is separate from the mechanical ventilator 10, for example being the microprocessor of a desktop computer—in these embodiments, the parameter estimation is performed at the desktop computer (or other device separate from the ventilator 10). In these embodiments, the microprocessor separate from the ventilator 10 may read the sensors 24, 26 directly, or the ventilator 10 may read the sensors 24, 26 and the desktop computer or other separate device acquires the measurements from the ventilator 10, e.g. via a USB or other wired or wireless digital communication connection. In these latter embodiments, the lung volume determination component 30 may optionally be implemented by a microprocessor of the ventilator 10 (or by an analog integration circuit), so that the desktop computer reads all of the values Pₐₒ(t), \dot{V}(t), and V(t) from the ventilator 10 via the USB or other connection.

The data processing components 30, 40 may also be implemented as a non-transitory storage medium storing instructions readable and executable by a microprocessor (e.g. as described above) to implement the disclosed functions. The non-transitory storage medium may, for example, comprise a read-only memory (ROM), programmable read-only memory (PROM), flash memory, or other respository of firmware for the ventilator 10. Additionally or alternatively, the non-transitory storage medium may comprise a computer hard drive (suitable for computer-implemented embodiments), an optical disk (e.g. for installation on such a computer), a network server data storage (e.g. RAID array) from which the ventilator 10 or a computer can download the system software or firmware via the Internet or another electronic data network, or so forth.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A medical ventilator system comprising:
a ventilator configured to deliver ventilation to a ventilated patient;
an airway pressure sensor configured to acquire airway pressure data for the ventilated patient;
an airway airflow sensor configured to acquire airway air flow data for the ventilated patient;
a probabilistic estimator module comprising a microprocessor programmed to estimate respiratory parameters of the ventilated patient by fitting a respiration system model to a data set comprising the acquired airway pressure data and the acquired airway air flow data using probabilistic analysis in which the respiratory parameters are represented as random variables; and
a display component configured to display the estimated respiratory parameters of the ventilated patient.

2. The medical ventilator system of claim 1 wherein the probabilistic estimator module estimates the respiratory parameters of the ventilated patient including at least respiratory system resistance and respiratory system compliance or elastance.

3. The medical ventilator system of claim 2 wherein the respiration system model is a first-order linear single-compartment model governed by the equation of motion:
Pₐₒ(t)=Rᵣₛ·\dot{V}(t)+Eᵣₛ·V(t)+P₀
where Rᵣₛ is the respiratory system resistance, Eᵣₛ is the respiratory system elastance or the inverse of the respiratory system compliance, Pₐₒ(t) is the airway pressure data, \dot{V}(t) is the airway air flow data, V(t) is lung volume data derived from \dot{V}(t) by an integration operation over time, Pₘᵤₛ(t) represents pressure generated by respiratory muscles of the ventilated patient, and P₀ represents pressure remaining in the lungs at the end of expiration.

4. The medical ventilator system of claim 3 wherein the ventilated patient is a passive patient for whom Pₘᵤₛ(t)=0 over the entire breath cycle.

5. The medical ventilator system of claim 1 wherein the probabilistic estimator module estimates the respiratory parameters of the ventilated patient by fitting the respiration system model using Bayesian analysis comprising computing a posterior parameter probability density function P(θ|Z) given by:
$$p ( θ \vert Z ) = \frac{p ( Z \vert θ ) \cdot p ( θ )}{p ( Z )}$$
where θ is a random variable representing the respiratory parameters to be estimated, Z represents the data set, p(Z) is a probability density function of Z, and p(θ) is a prior probability distribution function of θ.

6. The medical ventilator system of claim 5 further comprising:
a prior information repository storing prior information for the respiratory parameters to be estimated for a plurality of different patient classes,
wherein the probabilistic estimator module generates the prior probability distribution function P(θ) based on prior information from the prior information repository for a patient class to which the ventilated patient belongs.

7. The medical ventilator system of claim 6 wherein:
the respiratory parameters to be estimated include respiratory system resistance Rᵣₛ, respiratory system elastance Eᵣₛ, and pressure P₀ remaining in the lungs at the end of expiration; and
probabilistic estimator module generates the prior probability distribution function p(θ) according to:
p(θ)=p(Rᵣₛ)·p(Eᵣₛ)·p(P₀)
where p(Rᵣₛ) is a prior probability distribution function for Rᵣₛ obtained from the prior information repository for the patient class to which the ventilated patient belongs, p(Eᵣₛ) is a prior probability distribution function for Eᵣₛ obtained from the prior information repository for the patient class to which the ventilated patient belongs, and p(P₀) is a prior probability distribution function for P₀ obtained from the prior information repository for the patient class to which the ventilated patient belongs.

8. The medical ventilator system of claim 1 wherein the probabilistic estimator module estimates the respiratory parameters of the ventilated patient using probabilistic analysis including:
generating a probability density function for each respiratory parameter to be estimated; and
estimating each respiratory parameter to be estimated based on the probability density function generated for that respiratory parameter.

9. The medical ventilator system of claim 8 wherein the display component is configured to further display the generated probability density functions for the respiratory parameters to be estimated.

10. The medical ventilator system of claim 8 wherein the display component is configured to further display a confidence interval or uncertainty for each estimated respiratory parameter based on the probability density function generated for that respiratory parameter by the probabilistic estimator module.

11. A non-transitory storage medium storing instructions readable and executable by a microprocessor to perform a respiratory parameter estimation method comprising:
receiving a data set comprising airway pressure data Pₐₒ(t), airway air flow data \dot{V}(t), and lung volume data V(t) for a ventilated patient receiving ventilation from a mechanical ventilator; and
estimating respiratory parameters of the ventilated patient including at least respiratory system resistance Rᵣₛ and respiratory system compliance Cᵣₛ or elastance Eᵣₛ by fitting a respiration system model to the data set using Bayesian analysis in which the respiratory parameters are represented as probability density functions; and
causing an estimated respiratory parameter to be displayed on a display device.

12. The non-transitory storage medium of claim 11 wherein the respiration system model is a first-order linear single-compartment model.

13. The non-transitory storage medium of claim 11 wherein the respiratory parameters further include a pressure P₀ remaining in the lungs at the end of expiration.

14. The non-transitory storage medium of claim 11 wherein the Bayesian analysis estimates the respiratory parameters of the ventilated patient by computing a posterior parameter probability density function p(θ|Z) having the value:
$$p ( θ \vert Z ) = \frac{p ( Z \vert θ ) \cdot p ( θ )}{p ( Z )}$$
where θ represents the respiratory parameters to be estimated, Z represents the data set, p(Z) is a probability density function of Z, and p(θ) is a prior probability distribution function of θ.

15. The non-transitory storage medium of claim 14 wherein the respiratory parameter estimation method further comprises:
generating the prior probability distribution function p(θ) based on prior information for a patient class to which the ventilated patient belongs.

16. The non-transitory storage medium of claim 15 wherein generating the prior probability distribution function p(θ) includes:
receiving a prior probability distribution function for the patient class to which the ventilated patient belongs for each respiratory parameter to be estimated; and
generating the prior probability distribution function p(θ) as the product of the received prior probability distribution functions.

17. The non-transitory storage medium of claim 11 wherein the respiratory parameter estimation method further comprises:
causing the probability density function representing the displayed estimated respiratory parameter to be displayed together with the displayed estimated respiratory parameter on the display device.

18. The non-transitory storage medium of claim 11 wherein receiving the data set includes receiving the airway air flow data \dot{V}(t) and computing the lung volume data V(t) by integrating the airway air flow data \dot{V}(t) over time.

19. A medical ventilation method comprising:
ventilating a ventilated patient using a mechanical ventilator;
during the ventilating, acquiring a data set comprising airway pressure data Pₐₒ(t) and airway air flow data \dot{V}(t) for the ventilated patient;
using a microprocessor, estimating respiratory system resistance Rᵣₛ and respiratory system compliance Cᵣₛ or elastance Eᵣₛ by fitting a respiration system model to the acquired data set using probabilistic analysis in which the respiratory system resistance Rᵣₛ is represented by a probability density function and the respiratory system compliance Cᵣₛ or elastance Eᵣₛ is represented by a probability density function; and
displaying the estimated respiratory system resistance Rᵣₛ and respiratory system compliance Cᵣₛ or elastance Eᵣₛ on a display component.

20. The medical ventilator method of claim 19 wherein the probabilistic analysis is Bayesian analysis.
